(19) United States
(12) Patent Application Publication
(10) Pub. No.: US 2026/0306519 A1
(43) Pub. Date: Oct. 8, 2026

(54) REDUCING NECROTIC ENTERITIS EFFECT IN A BIRD USING AN OLIGOSACCHARIDE PREPARATION

(71) Applicant: DSM IP ASSETS B.V., Maastricht (NL)

(72) Inventors: Elkin AMAYA, Getzersdorf (AT); Britt BLOKKER, Getzersdorf (AT); Cristiano BORTOLUZZI, Getzersdorf (AT); John Michael GEREMIA, Getzersdorf (AT); Jose-Otavio SORBARA, Getzersdorf (AT)

(21) Appl. No.: 18/998,347

(22) PCT Filed: Jul. 27, 2023
(86) PCT No.: PCT/EP2023/070807

§ 371 Date: Jan. 24, 2025

Related U.S. Application Data
(60) Provisional application No. 63/393,325, filed on Jul. 29, 2022.

(51) Int. Cl. A61K 31/702; A61P 31/04

(52) U.S. Cl. CPC A61K 31/702; A61P 31/04

(57) ABSTRACT

The present invention relates to a method reducing necrotic enteritis effect in a bird comprising administering an oligosaccharide preparation, and to an oligosaccharide preparation for use in treatment, amelioration and/or prophylaxis of a bird suffering from necrotic enteritis or in risk thereof.

The present invention relates to a method for reducing necrotic enteritis effect in a bird and to a use of the oligosaccharide preparation.

Necrotic enteritis is one of the most common diseases in poultry farming, causing severe consequences for affected animals as well as financial losses for the agriculturist. Necrotic enteritis usually occurs in broiler chickens, but has also been described in other birds such as layer chickens or turkeys. In the event of a clinical outbreak, necrotic enteritis causes mortality rates of up to 50%. In addition, necrotic enteritis typically causes inter alia intestinal lesions, and negatively affects animal welfare as well as animal performance, such as reduced body weight gain or increased feed conversion ratio. Financial losses due to necrotic enteritis are estimated at five to six billion USD per year.

Necrotic enteritis is caused by atypically high intestinal growth of Clostridium perfringens. Under normal conditions, C. perfringens is a regular member of a bird's intestinal microbiome. However, under certain conditions, over proliferation of C. perfringens can be induced, accompanied with toxin production by C. perfringens. Conditions known to favor such over proliferation include consumption of feed contaminated with mycotoxin, coccidiosis stress from Eimeria contamination and/or vaccination, immune repression, low-quality protein, reduced amino acid digestibility or nutrient absorption, increased protein availability in the lumen, reduced gut integrity, protein leakage, or pathogen translocation.

Traditionally, C. perfringens-induced necrotic enteritis could be treated by administration of antibiotics. However in order to avoid spreading of bacterial mechanisms towards antibiotic resistance, the use of antibiotics in animal feed should be avoided as much as possible. Alternative measures to reduce the risk for necrotic enteritis outbreaks include finetuning of feed composition and administration. Despite such efforts, there is a need for further measures supporting mitigation of the negative effect(s) of necrotic enteritis. In view of the prior art as outlined above, it is therefore an objective of the present invention to provide such means and measures.

This objective is achieved by a method for reducing necrotic enteritis effect in a bird, comprising the step of administering an oligosaccharide preparation to a bird in need of reducing necrotic enteritis effect, wherein the oligosaccharide preparation comprises at least n fractions of oligosaccharides each having a distinct degree of polymerization selected from 1 to n, wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry. The at least n fractions of oligosaccharides each having a distinct degree of polymerization selected from 1 to n can be referred to as DP1 to DPn fractions. By providing such a method it is possible to reduce, mitigate or avoid one or more of the effects caused by necrotic enteritis. In some embodiments, such necrotic enteritis effect is reduced partially. In other embodiments, such necrotic enteritis effect may be eliminated entirely, i.e. the bird in need of reducing necrotic enteritis effect cannot be distinguished from a bird not in need of reducing necrotic enteritis effect.

In another aspect, the invention relates to a use of an oligosaccharide preparation for reducing necrotic enteritis effect in a bird in need thereof, wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry. In particular, said oligosaccharide preparation is used for administration to a bird in need of reducing necrotic enteritis effect.

In a further aspect, the invention relates to an oligosaccharide preparation for use in treatment, amelioration and/or prophylaxis of a bird suffering from necrotic enteritis and/or in risk thereof, wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90% (e.g. from 1% to 90%; or e.g. from about 0.5% to about 15%) of anhydro-subunit containing oligosaccharides by relative abundance as determined by mass spectrometry.

In some embodiments, the necrotic enteritis effect is at least one selected from the group of reduced body weight gain of the bird in need of reducing necrotic enteritis effect compared to a control bird not in need of reducing necrotic enteritis effect; increased feed conversion ratio of the bird in need of reducing necrotic enteritis effect compared to the control bird; increased mortality (in particular increased necrotic enteritis-related mortality) of the bird in need of reducing necrotic enteritis effect compared to the control bird; and increased lesion score of the bird in need of reducing necrotic enteritis effect compared to the control bird.

In some embodiments, the bird in need of reducing necrotic enteritis effect is challenged by Clostridium perfringens. Such challenge by Clostridium perfringens consists in an atypically high occurrence of intestinal Clostridium perfringens. In some embodiments, the C. perfringens challenge due to C. perfringens over proliferation is a result of one or more of the following: consumption of feed contaminated with mycotoxin(s), coccidiosis stress from Eimeria contamination and/or vaccination against coccidiosis, immune repression, intake of low-quality protein, reduced amino acid digestibility or nutrient absorption, increased protein availability in the lumen, reduced gut integrity, protein leakage, or pathogen translocation; thus causing a need for reducing necrotic enteritis effect.

In some embodiments, the bird is poultry, e.g. chicken, duck, goose, turkey, guinea fowl, pigeon, quail. In specific embodiments, the bird is chicken, preferably broiler chicken.

In some embodiments, the necrotic enteritis effect is reduced on at least one day within 42 days after a first administration of the oligosaccharide preparation, selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s). In particular embodiments, the necrotic enteritis effect is reduced when compared to the day on which the bird in need of reducing necrotic enteritis effect is first administered the oligosaccharide preparation (i.e. day 1). For instance, a reduction in necrotic enteritis effect is observed when comparing day 14 to day 1, day 21 to day 1, day 35 to day 1, and/or day 42 to day 1. Herein, “day 14” indicates the fourteenth day after the bird in need of reducing necrotic enteritis effect is first administered the oligosaccharide preparation (i.e. after day 1); “day 21” indicates the twenty-first day after the bird in need of reducing necrotic enteritis effect is first administered the oligosaccharide preparation (i.e. after day 1); etc.

In some embodiments, the oligosaccharide preparation is comprised in a nutritional composition administered to the bird.

In some embodiments, the oligosaccharide preparation is comprised in the nutritional composition at an inclusion rate of at least 50 ppm, e.g. at least 50, 70, 100, 150, 200, 300, 400, 500, 600, 700, 800, 900 ppm. In some specific embodiments, the oligosaccharide preparation is comprised in the nutritional composition at a concentration of at least 50 ppm (e.g at least 100, 150, 200, 220, 250, 300, 310, 350, 400 ppm) of dried glycans of the oligosaccharide preparation.

In some embodiments, the oligosaccharide preparation is administered to the bird before, after, and/or simultaneously with the diet of the bird in need of reducing necrotic enteritis effect, preferably simultaneously with the diet.

The step of administering an oligosaccharide preparation to a bird in need of reducing necrotic enteritis effect as described herein is intended to be construed as providing the oligosaccharide preparation (e.g. as a part comprised in a nutritional composition) to the bird for consumption by said bird. In other words, the oligosaccharide preparation is fed to the bird. In one embodiment, this administering/providing/feeding may be effected by providing the nutritional composition to the bird in an ad libitum feeding fashion.

In some embodiments, administering the oligosaccharide preparation comprises providing the oligosaccharide preparation to a bird such that the bird may ingest the oligosaccharide preparation at will. In such embodiments, the animal ingests at least some portion of the oligosaccharide preparation.

Also, the oligosaccharide preparation may be provided to the bird on any appropriate schedule. In some embodiments, the bird is provided the oligosaccharide preparation on a daily basis, on a weekly basis, on a monthly basis, on an every other day basis, for at least three days out of every week, or for at least seven days out of every month.

In some embodiments, the oligosaccharide preparation is administered to the bird multiple times in a day. For examples, in some embodiments, the oligosaccharide preparation is administered to the bird at least 1, 2, 3, 4, 5, 6, 7, 8, 9, or 10 times a day. In some embodiments, the oligosaccharide preparation is administered to the bird at most 1, 2, 3, 4, 5, 6, 7, 8, 9, or 10 times a day. In some embodiments, the oligosaccharide preparation is administered to the bird at least 1, 2, 3, 4, 5, 6, 7, 8, 9, 10, 15, or 20 times a week. In some embodiments, the oligosaccharide preparation is administered to the bird at most 1, 2, 3, 4, 5, 6, 7, 8, 9, 10, 15, or 20 times a week. In some embodiments, the oligosaccharide preparation is administered to the bird every day, every other day, every 3 days, every 4 days, every week, every other week, or every month.

In certain embodiments, the oligosaccharide preparation is administered to the bird at certain time during the day. For example, in certain embodiments, the oligosaccharide preparation is administered to the bird in the morning, in the afternoon, and/or in the evening, or any combination thereof.

In some embodiments, the bird is provided the nutritional composition during certain diet phases. For example, some birds are provided a starter diet, e.g. between 0 to 14 days of age. In other embodiments, a bird is provided a grower diet, e.g. between 15 to 28 days of age, between 15 to 35 days of age, or between 15 to 39 days of age. In still other embodiments, a bird is provided a finisher diet, e.g. between 29 to 35 days of age, between 36 to 42 days of age, or between 40 to 46 days of age.

The oligosaccharide preparation may be fed to individual birds or a bird population. For example, in some embodiments, where the bird is poultry, the oligosaccharide preparation may be fed to an individual poultry or a poultry population.

The oligosaccharide preparation may be provided to a bird in any appropriate form, including, for example, in solid form, in liquid form, or a combination thereof. Also in case the oligosaccharide preparation is provided as part of a nutritional composition, said nutritional composition may be provided in any appropriate form, including, for example, in solid form, in liquid form, or a combination thereof. In certain embodiments, the nutritional composition or the oligosaccharide preparation is a liquid, such as a syrup or a solution. In other embodiments, the nutritional composition or the oligosaccharide preparation is a solid, such as pellets or powder. In yet other embodiments, the nutritional composition or the oligosaccharide preparation may be fed to the bird in both liquid and solid components, such as in a mash.

In some embodiments of the invention, the oligosaccharide preparation referred to herein is a synthetic oligosaccharide preparation.

It is considered that the oligosaccharide preparation may be provided in the form of a powderous formulation comprising at least 20% (w/w) (e.g. at least 20, 25, 30, 35, 40, 45 etc. % w/w) of the oligosaccharide preparation as referred to herein; at least 25% (w/w) of a silica-based adsorbate (e.g. diatomaceous earth, amorphous precipitated silica) having an average particle size D of less than or equal to 3000 μm (e.g. 100-500, 200-500, 200-300 μm); and optionally 0-25% (w/w) of water and/or an auxiliary substance; wherein the % are based on the total weight of the powderous formulation. For instance, such a powderous formulation may comprise 30-70% (w/w) of the oligosaccharide preparation as referred to herein; 30-70% (w/w) of a silica based adsorbate (e.g. having an average particle size of at least 50 μm); and 0-21% (w/w) of water; wherein the % are based on the total weight of the powderous formulation. In some embodiments the oligosaccharide preparation is formulated as described in any one of Examples 22-26 and 33 of WO 2020/097458.

In some embodiments, n of the oligosaccharide preparation is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In some embodiments, at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

In some embodiments, the oligosaccharide preparation has a weight average molecular weight from about 300 to 5000 g/mol, e.g. from about 2000 to 2800 g/mol, 2100 to 2700 g/mol, 2200 to 2600 g/mol, 2300 to 2500 g/mol, or 2320 to 2420 g/mol; 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

In some embodiments, the relative abundance of oligosaccharides in each of the n fractions of the oligosaccharide preparation decreases monotonically with its degree of polymerization

Descriptions and methods of manufacturing oligosaccharide preparations according to the invention are described in WO 2020/097458 and WO 2016/007778, in particular in the Examples described therein, in particular in any one of Examples 1-7, 16-18 of WO 2020/097458 A1, in the methods described in paragraph [317], and/or in any one of Examples 73-77, 80-89, 97-99, 101-110 of WO 2016/007778 A1. Oligosaccharide preparations according to the invention are characterized by the step of heating an aqueous composition comprising one or more feed sugars and a catalyst to a temperature and for a time sufficient to induce polymerization. Such oligosaccharide preparations may be termed synthetic oligosaccharide preparations. In the following, oligosaccharide preparations as referred to herein are further specified.

Merely for the sake of clarity, the term “oligosaccharide preparation” may refer to a preparation that comprises one or more oligosaccharides.

As used herein, an “oligosaccharide” or “oligomer” may refer to a monosaccharide or a compound containing two or more monosaccharide subunits linked by glycosidic bonds. An “oligosaccharide” may also refer to an anhydro-monosaccharide or a compound containing two or more monosaccharide subunits, where at least one monosaccharide unit is replaced by an anhydro-subunit. An “oligosaccharide” may be optionally functionalized. As used herein, the term “oligosaccharide” encompasses all species of the oligosaccharide, wherein each of the monosaccharide subunit in the oligosaccharide is independently and optionally functionalized and/or replaced with its corresponding anhydro-monosaccharide subunit.

An “anhydro-subunit” may be a product of reversible thermal dehydration of a monosaccharide (or monosaccharide subunit) or a sugar caramelization product. For example, an “anhydro-subunit” may be an anhydro-monosaccharide such as anhydro-glucose. As another example, an “anhydro-subunit” may be linked with one or more regular or anhydro-monosaccharide subunits via glycosidic linkage.

An oligosaccharide may be characterized to contain two or more monosaccharide subunits linked by glycosidic bonds. In this regard, a “gluco-oligosaccharide” may refer to a glucose or a compound containing two or more glucose monosaccharide subunits linked by glycosidic bonds. A “gluco-oligosaccharide” may also refer to an anhydro-glucose or a compound containing two or more glucose monosaccharide subunits linked by glycosidic bonds, wherein at least one monosaccharide subunit is replaced with an anhydro-glucose subunit. Similarly, a “galacto-oligosaccharide” may refer to a galactose or a compound containing two or more galactose monosaccharide subunits linked by glycosidic bonds. A “galacto-oligosaccharide” may also refer to an anhydro-galactose or a compound containing two or more galactose monosaccharide subunits linked by glycosidic bonds, wherein at least one monosaccharide subunit is replaced with an anhydro-galactose subunit. Analogously, a gluco-galactose-oligosaccharide may be a gluco-oligosaccharide, a galacto-oligosaccharide, or a compound containing one or more glucose monosaccharide subunits and one or more galactose monosaccharide subunits linked by glycosidic bonds, wherein at least one of the monosaccharide subunits is replaced with its respective anhydro-monosaccharide subunit. A gluco-galacto-xylo-oligosaccharide may refer to a compound produced by the condensation reaction of glucose, galactose, and xylose. An oligosaccharide preparation comprising gluco-galacto-xylo-oligosaccharides may comprise gluco-galactose-oligosaccharides, gluco-xylo-oligosaccharides, galacto-xylo-oligosaccharides, and compounds containing one or more glucose monosaccharide subunits, one or more xylose monosaccharide subunits, and one or more galactose monosaccharide subunits linked by glycosidic bonds.

As used herein, the term “monosaccharide unit” and “monosaccharide subunit” may be used interchangeably, unless suggested otherwise. A “monosaccharide subunit” may refer to a monosaccharide monomer in an oligosaccharide. For an oligosaccharide having a degree of polymerization of 1, the oligosaccharide may be referred to as a monosaccharide subunit or monosaccharide. For an oligosaccharide having a degree of polymerization higher than 1, its monosaccharide subunits are linked via glycosidic bonds.

As used herein, the term “regular monosaccharide” may refer to a monosaccharide that does not contain an anhydro-subunit. The term “regular disaccharide” may refer to a disaccharide that does not contain an anhydro-subunit. Accordingly, the term “regular subunit” may refer to a subunit that is not an anhydro-subunit.

The term “relative abundance” or “abundance,” as used herein, may refer to the abundance of a species in terms of how common or rare the species exists. For example, a DP1 fraction comprising 10% anhydro-subunit containing oligosaccharides by relative abundance may refer to a plurality of DP1 oligosaccharides, wherein 10%, by number, of the DP1 oligosaccharides are anhydro-monosaccharides.

Degree of Polymerization (DP) Distribution: A distribution of the degree of polymerization of the oligosaccharide preparation may be determined by any suitable analytical method and instrumentation, including but not limited to end group method, osmotic pressure (osmometry), ultracentrifugation, viscosity measurements, light scattering method, size exclusion chromatography (SEC), SEC-MALLS, field flow fractionation (FFF), asymmetric flow field flow fractionation (A4F), high-performance liquid chromatography (HPLC), and mass spectrometry (MS). For example, the distribution of the degree of polymerization may be determined and/or detected by mass spectrometry, such as MALDI-MS, LC-MS, or GC-MS. For another example, the distribution of the degree of polymerization may be determined and/or detected by SEC, such as gel permeation chromatography (GPC). As yet another example, the distribution of the degree of polymerization may be determined and/or detected by HPLC, FFF, or A4F. In another example, the degree of polymerization of the oligosaccharide preparation may be determined based on its molecular weight and molecular weight distribution (for a more detailed description see WO 2020/097458).

Anhydro-subunit Level: In some embodiments, each of the n fractions of oligosaccharides of the oligosaccharide preparation as described herein independently comprises an anhydro-subunit level. For instance, in some embodiments, the DP1 fraction comprises 10% anhydro-subunit containing oligosaccharides by relative abundance, and the DP2 fraction comprises 15% anhydro-subunit containing oligosaccharides by relative abundance. For another example, in some embodiments, DP1, DP2, and DP3 fraction each comprises 5%, 10%, and 2% anhydro-subunit containing oligosaccharides by relative abundance, respectively. In other embodiments, two or more fractions of oligosaccharides may comprise similar level of anhydro-subunit containing oligosaccharides. For example, in some embodiments, the DP1 and DP3 fraction each comprises about 5% anhydro-subunit containing oligosaccharides by relative abundance.

The level of anhydro-subunits may be determined by any suitable analytical methods, such as nuclear magnetic resonance (NMR) spectroscopy, mass spectrometry, HPLC, FFF, A4F, or any combination thereof. In some embodiments, the level of anhydro-subunits is determined, at least in part, by mass spectrometry such as MALDI-MS. In some embodiments, the level of anhydro-subunits may be determined, at least in part, by NMR. In some embodiments, the level of anhydro-subunits may be determined, at least in part, by HPLC. For example, in some embodiments, the level of anhydro-subunits may be determined by MALDI-MS, as illustrated in more detail in WO 2020/097458.

Glycosidic Linkages: In some embodiments, the oligosaccharide preparation described herein comprise a variety of glycosidic linkages. The type and distribution of the glycosidic linkages may depend on the source and manufacturing method of the oligosaccharide preparation. In some embodiments, the type and distribution of various glycosidic linkages may be determined and/or detected by any suitable methods known in the art such as NMR. For example, in some embodiments, the glycosidic linkages are determined and/or detected by proton NMR, carbon NMR, 2D NMR such as 2D JRES, HSQC, HMBC, DOSY, COSY, ECOSY, TOCSY, NOESY, or ROESY, or any combination thereof. In some embodiments, the glycosidic linkages are determined and/or detected, at least in part, by proton NMR. In some embodiments, the glycosidic linkages are determined and/or detected, at least in part, by carbon NMR. In some embodiments, the glycosidic linkages are determined and/or detected, at least in part, by 2D HSQC NMR.

In some embodiments, an oligosaccharide preparation may comprise one or more α-(1,2) glycosidic linkages, α-(1,3) glycosidic linkages, α-(1,4) glycosidic linkages, α-(1,6) glycosidic linkages, β-(1,2) glycosidic linkages, β-(1,3) glycosidic linkages, β-(1,4) glycosidic linkages, β-(1,6) glycosidic linkages, a (1,1) a glycosidic linkages, a (1,1) β glycosidic linkages, β (1,1) β glycosidic linkages, or any combination thereof.

In some embodiments, the oligosaccharide preparations have a glycosidic bond type distribution of about from 0 to 60 mol %, 5 to 55 mol %, 5 to 50 mol %, 5 to 45 mol %, 5 to 40 mol %, 5 to 35 mol %, 5 to 30 mol %, 5 to 25 mol %, 10 to 60 mol %, 10 to 55 mol %, 10 to 50 mol %, 10 to 45 mol %, 10 to 40 mol %, 10 to 35 mol %, 15 to 60 mol %, 15 to 55 mol %, 15 to 50 mol %, 15 to 45 mol %, 15 to 40 mol %, 15 to 35 mol %, 20 to 60 mol %, 20 to 55 mol %, 20 to 50 mol %, 20 to 45 mol %, 20 to 40 mol %, 20 to 35 mol %, 25 to 60 mol %, 25 to 55 mol %, 25 to 50 mol %, 25 to 45 mol %, 25 to 40 mol %, or 25 to 35 mol % of α-(1,6) glycosidic linkages.

Molecular Weight: The molecular weight and molecular weight distribution of the oligosaccharide preparation may be determined by any suitable analytical means and instrumentation, such as end group method, osmotic pressure (osmometry), ultracentrifugation, viscosity measurements, light scattering method, SEC, SEC-MALLS, FFF, A4F, HPLC, and mass spectrometry. In some embodiments, the molecular weight and molecular weight distribution are determined by mass spectrometry, such as MALDI-MS, LC-MS, or GC-MS. In some embodiments, the molecular weight and molecular weight distribution are determined by size exclusion chromatography (SEC), such as gel permeation chromatography (GPC). In other embodiments, the molecular weight and molecular weight distribution are determined by HPLC. In some embodiments, the molecular weight and molecular weight distribution are determined by MALDI-MS.

In some embodiments, the weight average molecular weight of the oligosaccharide preparation is about from 100 to 10000 g/mol, 200 to 8000 g/mol, 300 to 5000 g/mol, 500 to 5000 g/mol, 700 to 5000 g/mol, 900 to 5000 g/mol, 1100 to 5000 g/mol, 1300 to 5000 g/mol, 1500 to 5000 g/mol, 1700 to 5000 g/mol, 300 to 4500 g/mol, 500 to 4500 g/mol, 700 to 4500 g/mol, 900 to 4500 g/mol, 1100 to 4500 g/mol, 1300 to 4500 g/mol, 1500 to 4500 g/mol, 1700 to 4500 g/mol, 1900 to 4500 g/mol, 300 to 4000 g/mol, 500 to 4000 g/mol, 700 to 4000 g/mol, 900 to 4000 g/mol, 1100 to 4000 g/mol, 1300 to 4000 g/mol, 1500 to 4000 g/mol, 1700 to 4000 g/mol, 1900 to 4000 g/mol, 300 to 3000 g/mol, 500 to 3000 g/mol, 700 to 3000 g/mol, 900 to 3000 g/mol, 1100 to 3000 g/mol, 1300 to 3000 g/mol, 1500 to 3000 g/mol, 1700 to 3000 g/mol, 1900 to 3000 g/mol, 2100 to 3000 g/mol, 300 to 2500 g/mol, 500 to 2500 g/mol, 700 to 2500 g/mol, 900 to 2500 g/mol, 1100 to 2500 g/mol, 1300 to 2500 g/mol, 1500 to 2500 g/mol, 1700 to 2500 g/mol, 1900 to 2500 g/mol, 2100 to 2500 g/mol, 300 to 1500 g/mol, 500 to 1500 g/mol, 700 to 1500 g/mol, 900 to 1500 g/mol, 1100 to 1500 g/mol, 1300 to 1500 g/mol, 2000-2800 g/mol, 2100-2700 g/mol, 2200-2600 g/mol, 2300-2500 g/mol, or 2320-2420 g/mol. In some embodiments, the weight average molecular weight of the oligosaccharide preparation is about from 2000 to 2800 g/mol, 2100 to 2700 g/mol, 2200 to 2600 g/mol, 2300 to 2500 g/mol, or 2320 to 2420 g/mol.

Types of Oligosaccharides: In some embodiments, the species of oligosaccharides present in an oligosaccharide preparation referred to herein may depend on the type of the one or more feed sugars. For example, in some embodiments, the oligosaccharide preparations comprise a gluco-oligosaccharide when the feed sugars comprise glucose. For example, in some embodiments, the oligosaccharide preparations comprise a galacto-oligosaccharide when the feed sugars comprise galactose. For another example, in some embodiments, the oligosaccharide preparations comprise gluco-galacto-oligosaccharides when the feed sugars comprise galactose and glucose.

In some embodiments, the oligosaccharide preparations comprise one or more species of monosaccharide subunits. In some embodiments, the oligosaccharide preparation may comprise oligosaccharides with 1, 2, 3, 4, 5, 6, 7, 8, 9, 10, 11, 12, 13, 14, 15, 16, 17, 18, 19, 20, or more different species of monosaccharides subunits.

Method of Manufacturing Oligosaccharide Preparations: The method of manufacturing an oligosaccharide preparation according to the invention is described in detail in WO 2020/097458, comprising heating an aqueous composition comprising one or more feed sugars and a catalyst to a temperature and for a time sufficient to induce polymerization, wherein the catalyst is selected from the group consisting of: (+)-camphor-10-sulfonic acid; 2-pyridinesulfonic acid; 3-pyridinesulfonic acid; 8-hydroxy-5-quinolinesulfonic acid hydrate; α-hydroxy-2-pyridinemethanesulfonic acid; (B)-camphor-10-sulfonic acid; butylphosphonic acid; diphenylphosphinic acid; hexylphosphonic acid; methylphosphonic acid; phenylphosphinic acid; phenylphosphonic acid; tert-butylphosphonic acid; SS)-VAPOL hydrogenphosphate; 6-quinolinesulfonic acid, 3-(1-pyridinio)-1-propanesulfonate; 2-(2-pyridinyl) ethanesulfonic acid; 3-(2-pyridyl)-5,6-diphenyl-1,2,4-triazine-p,p′-disulfonic acid monosodium salt hydrate; 1,1′-binaphthyl-2,2′-diyl-hydrogenphosphate; bis(4-methoxyphenyl) phosphinic acid; phenyl (3,5-xylyl) phosphinic acid; L-cysteic acid monohydrate; poly(styrene sulfonic acid-co-divinylbenzene); lysine; Ethanedisulfonic acid; Ethanesulfonic acid; Isethionic acid; Homocysteic acid; HEPBS (N-(2-Hydroxyethyl) piperazine-N′-(4-butanesulfonic acid)); HEPES (4-(2-hydroxyethyl)-1-piperazineethanesulfonic acid); 2-Hydroxy-3-morpholinopropanesulfonic acid; 2-(N-morpholino) ethanesulfonic acid; Methanesulfonic acid; Methaniazide; Naphthalene-1-sulfonic acid; Naphthalene-2-sulfonic acid; Perfluorobutanesulfonic acid; 6-sulfoquinovose; Triflic acid; 2-aminoethanesulfonic acid; Benzoic acid; Chloroacetic acid; Trifluoroacetic acid; Caproic acid; Enanthic acid; Caprylic acid; Pelargonic acid; Lauric acid; Pamitic acid; Stearic acid; Arachidic acid; Aspartic acid; Glutamic acid; Serine; Threonine; Glutamine; Cysteine; Glycine; Proline; Alanine; Valine; Isoleucine; Leucine; Methionine; Phenylalanine; Tyrosine; Tryptophan.

In some embodiments, the polymerization of the feed sugars is achieved by a step-growth polymerization. In some embodiments, the polymerization of the feed sugars is achieved by polycondensation.

Feed Sugar: The one or more feed sugars used in the methods of manufacturing oligosaccharide preparations described herein may comprise one or more types of sugars. In some embodiments, the one or more feed sugars comprise monosaccharides, disaccharides, trisaccharides, tetrasaccharides, or any mixtures thereof.

In some embodiments, the one or more feed sugars comprise glucose. In some embodiments, the one or more feed sugars comprise glucose and galactose. In some embodiments, the one or more feed sugars comprise glucose, xylose, and galactose. In some embodiments, the one or more feed sugars comprise glucose and mannose. In some embodiments, the one or more feed sugars comprise glucose and fructose. In some embodiments, the one or more feed sugars comprise glucose, fructose, and galactose. In some embodiments, the one or more feed sugars comprise glucose, galactose, and mannose.

As used herein, the singular forms “a,” “and,” and “the” include plural referents unless the context clearly dictates otherwise. Thus, for example, reference to “an agent” includes a plurality of such agents, and reference to “the oligosaccharide” includes reference to one or more oligosaccharides (or to a plurality of oligosaccharides) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The invention is further characterized by the following items:
Item 1: Method for reducing necrotic enteritis effect in a bird, comprising the step of administering an oligosaccharide preparation to a bird in need of reducing necrotic enteritis effect, wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90%, e.g. from 1% to 90%, or e.g. from about 0.5% to about 15%, of anhydro-subunit containing oligosaccharides by relative abundance as determined by mass spectrometry.
Item 2: The method of item 1, wherein the necrotic enteritis effect is at least one selected from the group of reduced body weight gain of the bird in need of reducing necrotic enteritis effect compared to a control bird not in need of reducing necrotic enteritis effect; increased feed conversion ratio of the bird in need of reducing necrotic enteritis effect compared to the control bird; increased mortality of the bird in need of reducing necrotic enteritis effect compared to the control bird; and increased lesion score of the bird in need of reducing necrotic enteritis effect compared to the control bird.
Item 3: The method of item 1 or 2, wherein the bird in need of reducing necrotic enteritis effect is challenged by Clostridium perfringens.
Item 4: The method of any one of the preceding items, wherein the oligosaccharide preparation is comprised in a nutritional composition administered to the bird.
Item 5: The method of any one of the preceding items, wherein the oligosaccharide preparation is comprised in the nutritional composition at an inclusion rate of at least 50 ppm, e.g. at least 50, 70, 100, 150, 200, 300, 400, 500, 600, 700, 800, 900 ppm.
Item 6: The method of any one of the preceding items, wherein the oligosaccharide preparation is administered to the bird before, after, and/or simultaneously with the diet of the bird in need of reducing necrotic enteritis effect, preferably simultaneously with the diet.
Item 7: The method of any one of the preceding items, wherein the bird is poultry, e.g. chicken, duck, goose, turkey, guinea fowl, pigeon, quail; preferably chicken.
Item 8: The method of any one of the preceding items, wherein the necrotic enteritis effect is reduced on at least one day within 42 days after a first administration of the oligosaccharide preparation, selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s).
Item 9: The method of any one of the preceding items, wherein n of the oligosaccharide preparation is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.
Item 10: The method of any one of the preceding items, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 11: The method of any one of the preceding items, wherein the oligosaccharide preparation has a weight average molecular weight from about 300 to 5000 g/mol, e.g. from about 2000 to 2800 g/mol, 2100 to 2700 g/mol, 2200 to 2600 g/mol, 2300 to 2500 g/mol, or 2320 to 2420 g/mol; 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.
Item 12: The method of any one of the preceding items, wherein the relative abundance of oligosaccharides in each of the n fractions of the oligosaccharide preparation decreases monotonically with its degree of polymerization.
Item 13: The method according to any one of the preceding items, wherein the relative abundance of oligosaccharides in at least 5, 10, 20, or 30 DP fractions of the oligosaccharide preparation decreases monotonically with its degree of polymerization.
Item 14: The method according to any one of the preceding items, wherein the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 15: The method according to any one of the preceding items, wherein each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 16: The method according to any one of the preceding items, wherein at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 40%, 50%, 60%, 70%, or 80% anhydro-subunit containing oligosaccharides by relative abundance.
Item 17: The method according to any one of the preceding items, wherein the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, 30%, 40%, 50%, 60%, 70%, or 80% anhydro-subunit containing oligosaccharides by relative abundance.
Item 18: The method according to any one of the preceding items, wherein each fraction of the oligosaccharide preparation comprises greater than 20%, 21%, 22%, 23%, 24%, or 25% anhydro-subunit containing oligosaccharides by relative abundance.
Item 19: The method according to any one of the preceding items, wherein more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit.
Item 20: The method according to any one of the preceding items, wherein the oligosaccharide preparation has a DP1 fraction content from 1 to 40% by relative abundance.
Item 21: The method according to any one of the preceding items, wherein the oligosaccharide preparation has a DP2 fraction content from 1 to 35% by relative abundance.
Item 22: The method according to any one of the preceding items, wherein the oligosaccharide preparation has a DP3 fraction content from 1 to 30% by relative abundance.
Item 23: The method according to any one of the preceding items, wherein the oligosaccharide preparation has a DP4 fraction content from 0.1 to 20% by relative abundance.
Item 24: The method according to any one of the preceding items, wherein the oligosaccharide preparation has a DP5 fraction content from 0.1 to 15% by relative abundance.
Item 25: The method according to any one of the preceding items, wherein the ratio of DP2 fraction to DP1 fraction of the oligosaccharide preparation is 0.02-0.40 by relative abundance.
Item 26: The method according to any one of the preceding items, wherein the ratio of DP3 fraction to DP2 fraction of the oligosaccharide preparation is 0.01-0.30 by relative abundance.
Item 27: The method according to any one of the preceding items, wherein the aggregate content of DP1 and DP2 fractions in the oligosaccharide preparation is less than 50, 30, or 10% by relative abundance.
Item 28: The method according to any one of the preceding items, wherein the oligosaccharide preparation comprises at least 103, 104, 105, 106 or 109 different oligosaccharide species.
Item 29: The method according to any one of the preceding items, wherein two or more independent oligosaccharides of the oligosaccharide preparation comprise different anhydro-subunits.
Item 30: The method according to any one of the preceding items, wherein the oligosaccharide preparation comprises one or more anhydro-subunits that are products of reversible thermal dehydration of monosaccharides.
Item 31: The method according to any one of the preceding items, wherein the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits.
Item 32: The method according to any one of the preceding items, wherein the oligosaccharide preparation comprises one or more anhydro-glucose, anhydro-galactose, anhydro-mannose, or anhydro-fructose subunits.
Item 33: The method according to any one of the preceding items, wherein the oligosaccharide preparation comprises one or more 1,6-anhydro-β-D-glucofuranose or 1,6-anhydro-β-D-glucopyranose subunits. In some embodiments, the oligosaccharide preparation comprises both 1,6-anhydro-β-D-glucofuranose and 1,6-anhydro-β-D-glucopyranose anhydro-subunits.
Item 34: The method according to any one of the preceding items, wherein a ratio of 1,6-anhydro-β-D-glucofuranose to 1,6-anhydro-β-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation.
Item 35: The method according to any one of the preceding items, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation.
Item 36: The method according to any one of the preceding items, wherein the ratio of 1,6-anhydro-β-D-glucofuranose to 1,6-anhydro-β-D-glucopyranose is about 2:1 in the oligosaccharide preparation.
Item 37: The method according to any one of the preceding items, wherein the ratio of 1,6-anhydro-β-D-glucofuranose to 1,6-anhydro-3-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation.
Item 38: The method according to any one of the preceding items, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation.
Item 39: The method according to any one of the preceding items, wherein the ratio of 1,6-anhydro-β-D-glucofuranose to 1,6-anhydro-β-D-glucopyranose is about 2:1 in each fraction of the oligosaccharide preparation.
Item 40: The method according to any one of the preceding items, wherein at least 1%, 5%, 10%, 20%, 30%, 40%, 50%, 60%, 70%, 80%, or 90% of anhydro-subunits in the oligosaccharide preparation are selected from a group consisting of 1,6-anhydro-β-D-glucofuranose and 1,6-anhydro-β-D-glucopyranose.
Item 41: The method according to any one of the preceding items, wherein the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.
Item 42: The method according to any one of the preceding items, wherein the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol.
Item 43: The method according to any one of the preceding items, wherein the distribution of the degree of polymerization is determined and/or detected by MALDI-MS, GC-MS, LC-MS, SEC, HPLC and/or combination(s) thereof (e.g. MALDI-MS and SEC).
Item 44: The method according to any one of the preceding items, wherein the degree of polymerization of the oligosaccharide preparation may be determined based on its molecular weight and molecular weight distribution.
Item 45: The method according to any one of the preceding items, wherein the oligosaccharide preparation is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed); and/or wherein the oligosaccharide preparation comprised in the nutritional composition at an inclusion rate of at least 50 ppm (e.g. at least 50, 70, 100, 150, 200, 300, 400, 500 ppm); and/or wherein the oligosaccharide preparation comprised in the nutritional composition at a concentration of at least 50 ppm (e.g. at least 50, 70, 100, 150, 200, 300, 400, 500 ppm).
Item 46: The method according to any one of the preceding items, wherein the oligosaccharide preparation is administered for at least one day, preferably for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, or 245 days, most preferably, the nutritional composition is administered continuously, i.e. uninterruptedly.
Item 47: Use of an oligosaccharide preparation for reducing necrotic enteritis effect in a bird in need thereof, wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90%, e.g. from 1% to 90%, or e.g. from about 0.5% to about 15%, of anhydro-subunit containing oligosaccharides by relative abundance as determined by mass spectrometry.
Item 48: The use of item 47, wherein n of the oligosaccharide preparation is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.
Item 49: The use of item 47 or 48, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 50: The use of any one of items 47-49, wherein the oligosaccharide preparation has a weight average molecular weight from about 300 to 5000 g/mol, e.g. from about 2000 to 2800 g/mol, 2100 to 2700 g/mol, 2200 to 2600 g/mol, 2300 to 2500 g/mol, or 2320 to 2420 g/mol; 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.
Item 51: The use of any one of items 47-50, wherein the relative abundance of oligosaccharides in each of the n fractions of the oligosaccharide preparation decreases monotonically with its degree of polymerization.
Item 52: The use of any one of items 47-51, wherein the relative abundance of oligosaccharides in at least 5, 10, 20, or 30 DP fractions of the oligosaccharide preparation decreases monotonically with its degree of polymerization.
Item 53: The use of any one of items 47-52, wherein the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 54: The use of any one of items 47-53, wherein each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 55: The use of any one of items 47-54, wherein at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 40%, 50%, 60%, 70%, or 80% anhydro-subunit containing oligosaccharides by relative abundance.
Item 56: The use of any one of items 47-55, wherein the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, 30%, 40%, 50%, 60%, 70%, or 80% anhydro-subunit containing oligosaccharides by relative abundance.
Item 57: The use of any one of items 47-56, wherein each fraction of the oligosaccharide preparation comprises greater than 20%, 21%, 22%, 23%, 24%, or 25% anhydro-subunit containing oligosaccharides by relative abundance.
Item 58: The use of any one of items 47-57, wherein more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit.
Item 59: The use of any one of items 47-58, wherein the oligosaccharide preparation has a DP1 fraction content from 1 to 40% by relative abundance.
Item 60: The use of any one of items 47-59, wherein the oligosaccharide preparation has a DP2 fraction content from 1 to 35% by relative abundance.
Item 61: The use of any one of items 47-60, wherein the oligosaccharide preparation has a DP3 fraction content from 1 to 30% by relative abundance.
Item 62: The use of any one of items 47-61, wherein the oligosaccharide preparation has a DP4 fraction content from 0.1 to 20% by relative abundance.
Item 63: The use of any one of items 47-62, wherein the oligosaccharide preparation has a DP5 fraction content from 0.1 to 15% by relative abundance.
Item 64: The use of any one of items 47-63, wherein the ratio of DP2 fraction to DP1 fraction of the oligosaccharide preparation is 0.02-0.40 by relative abundance.
Item 65: The use of any one of items 47-64, wherein the ratio of DP3 fraction to DP2 fraction of the oligosaccharide preparation is 0.01-0.30 by relative abundance.
Item 66: The use of any one of items 47-65, wherein the aggregate content of DP1 and DP2 fractions in the oligosaccharide preparation is less than 50, 30, or 10% by relative abundance.
Item 67: The use of any one of items 47-66, wherein the oligosaccharide preparation comprises at least 103, 104, 105, 106 or 109 different oligosaccharide species.
Item 68: The use of any one of items 47-67, wherein two or more independent oligosaccharides of the oligosaccharide preparation comprise different anhydro-subunits.
Item 69: The use of any one of items 47-68, wherein the oligosaccharide preparation comprises one or more anhydro-subunits that are products of reversible thermal dehydration of monosaccharides.
Item 70: The use of any one of items 47-69, wherein the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits.
Item 71: The use of any one of items 47-70, wherein the oligosaccharide preparation comprises one or more anhydro-glucose, anhydro-galactose, anhydro-mannose, or anhydro-fructose subunits.
Item 72: The use of any one of items 47-71, wherein the oligosaccharide preparation comprises one or more 1,6-anhydro-β-D-glucofuranose or 1,6-anhydro-β-D-glucopyranose subunits. In some embodiments, the oligosaccharide preparation comprises both 1,6-anhydro-β-D-glucofuranose and 1,6-anhydro-β-D-glucopyranose anhydro-subunits.
Item 73: The use of any one of items 47-72, wherein a ratio of 1,6-anhydro-β-D-glucofuranose to 1,6-anhydro-β-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation.
Item 74: The use of any one of items 47-73, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation.
Item 75: The use of any one of items 47-74, wherein the ratio of 1,6-anhydro-β-D-glucofuranose to 1,6-anhydro-β-D-glucopyranose is about 2:1 in the oligosaccharide preparation.
Item 76: The use of any one of items 47-75, wherein the ratio of 1,6-anhydro-β-D-glucofuranose to 1,6-anhydro-β-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation.
Item 77: The use of any one of items 47-76, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation.
Item 78: The use of any one of items 47-77, wherein the ratio of 1,6-anhydro-β-D-glucofuranose to 1,6-anhydro-β-D-glucopyranose is about 2:1 in each fraction of the oligosaccharide preparation.
Item 79: The use of any one of items 47-78, wherein at least 1%, 5%, 10%, 20%, 30%,
40%, 50%, 60%, 70%, 80%, or 90% of anhydro-subunits in the oligosaccharide preparation are selected from a group consisting of 1,6-anhydro-β-D-glucofuranose and 1,6-anhydro-β-D-glucopyranose.
Item 80: The use of any one of items 47-79, wherein the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.
Item 81: The use of any one of items 47-80, wherein the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol.
Item 82: The use of any one of items 47-81, wherein the distribution of the degree of polymerization is determined and/or detected by MALDI-MS, GC-MS, LC-MS, SEC, HPLC and/or combination(s) thereof (e.g. MALDI-MS and SEC).
Item 83: The use of any one of items 47-84, wherein the degree of polymerization of the oligosaccharide preparation may be determined based on its molecular weight and molecular weight distribution.
Item 84: The use of any one of items 47-83, wherein the oligosaccharide preparation is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed); and/or wherein the oligosaccharide preparation comprised in the nutritional composition at an inclusion rate of at least 50 ppm (e.g. at least 50, 70, 100, 150, 200, 300, 400, 500 ppm); and/or wherein the oligosaccharide preparation comprised in the nutritional composition at a concentration of at least 50 ppm (e.g. at least 50, 70, 100, 150, 200, 300, 400, 500 ppm).
Item 85: The use of any one of items 47-84, wherein the oligosaccharide preparation is administered for at least one day, preferably for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, or 245 days, most preferably, the nutritional composition is administered continuously, i.e. uninterruptedly.
Item 86: Oligosaccharide preparation for use in treatment, amelioration and/or prophylaxis of a bird suffering from necrotic enteritis and/or in risk thereof, wherein the oligosaccharide preparation comprises at least n fractions of oligosaccharides each having a distinct degree of polymerization selected from 1 to n (DP1 to DPn fractions), wherein n is an integer greater than or equal to 2; and wherein each fraction comprises from at least about 0.5% to about 90%, e.g. from 1% to 90%, or e.g. from about 0.5% to about 15%, of anhydro-subunit containing oligosaccharides by relative abundance as determined by mass spectrometry.
Item 87: The oligosaccharide preparation for use in treatment, amelioration and/or prophylaxis of a bird suffering from necrotic enteritis and/or in risk thereof of item 86, wherein n of the oligosaccharide preparation is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.
Item 88: The oligosaccharide preparation for use in treatment, amelioration and/or prophylaxis of a bird suffering from necrotic enteritis and/or in risk thereof of item 86 or item 87, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.
Item 89: The oligosaccharide preparation for use in treatment, amelioration and/or prophylaxis of a bird suffering from necrotic enteritis and/or in risk thereof of any one of items 86-88, wherein the oligosaccharide preparation has a weight average molecular weight from about 300 to 5000 g/mol, e.g. from about 2000 to 2800 g/mol, 2100 to 2700 g/mol, 2200 to 2600 g/mol, 2300 to 2500 g/mol, or 2320 to 2420 g/mol; 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.
Item 90: The oligosaccharide preparation for use in treatment, amelioration and/or prophylaxis of a bird suffering from necrotic enteritis and/or in risk thereof of any one of items 86-89, wherein the relative abundance of oligosaccharides in each of the n fractions of the oligosaccharide preparation decreases monotonically with its degree of polymerization.
Item 91: The oligosaccharide preparation for use in treatment, amelioration and/or prophylaxis of a bird suffering from necrotic enteritis and/or in risk thereof of any one of items 86-90, wherein the relative abundance of oligosaccharides in at least 5, 10, 20, or 30 DP fractions of the oligosaccharide preparation decreases monotonically with its degree of polymerization.
Item 92: The oligosaccharide preparation for use in treatment, amelioration and/or prophylaxis of a bird suffering from necrotic enteritis and/or in risk thereof of any one of items 86-91, wherein the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 93: The oligosaccharide preparation for use in treatment, amelioration and/or prophylaxis of a bird suffering from necrotic enteritis and/or in risk thereof of any one of items 86-92, wherein each fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance.
Item 94: The oligosaccharide preparation for use in treatment, amelioration and/or prophylaxis of a bird suffering from necrotic enteritis and/or in risk thereof of any one of items 86-93, wherein at least one fraction of the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 40%, 50%, 60%, 70%, or 80% anhydro-subunit containing oligosaccharides by relative abundance.
Item 95: The oligosaccharide preparation for use in treatment, amelioration and/or prophylaxis of a bird suffering from necrotic enteritis and/or in risk thereof of any one of items 86-94, wherein the oligosaccharide preparation comprises greater than 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%, 30%, 40%, 50%, 60%, 70%, or 80% anhydro-subunit containing oligosaccharides by relative abundance.
Item 96: The oligosaccharide preparation for use in treatment, amelioration and/or prophylaxis of a bird suffering from necrotic enteritis and/or in risk thereof of any one of items 86-95, wherein each fraction of the oligosaccharide preparation comprises greater than 20%, 21%, 22%, 23%, 24%, or 25% anhydro-subunit containing oligosaccharides by relative abundance.
Item 97: The oligosaccharide preparation for use in treatment, amelioration and/or prophylaxis of a bird suffering from necrotic enteritis and/or in risk thereof of any one of items 86-96, wherein more than 99%, 95%, 90%, 85%, 80%, 75%, 70%, 65%, 60%, 55%, 50%, 45%, 40%, 35%, or 30% of the anhydro-subunit containing oligosaccharides of the oligosaccharide preparation have only one anhydro-subunit.
Item 98: The oligosaccharide preparation for use in treatment, amelioration and/or prophylaxis of a bird suffering from necrotic enteritis and/or in risk thereof of any one of items 86-97, wherein the oligosaccharide preparation has a DP1 fraction content from 1 to 40% by relative abundance.
Item 99: The oligosaccharide preparation for use in treatment, amelioration and/or prophylaxis of a bird suffering from necrotic enteritis and/or in risk thereof of any one of items 86-98, wherein the oligosaccharide preparation has a DP2 fraction content from 1 to 35% by relative abundance.
Item 100: The oligosaccharide preparation for use in treatment, amelioration and/or prophylaxis of a bird suffering from necrotic enteritis and/or in risk thereof of any one of items 86-99, wherein the oligosaccharide preparation has a DP3 fraction content from 1 to 30% by relative abundance.
Item 101: The oligosaccharide preparation for use in treatment, amelioration and/or prophylaxis of a bird suffering from necrotic enteritis and/or in risk thereof of any one of items 86-100, wherein the oligosaccharide preparation has a DP4 fraction content from 0.1 to 20% by relative abundance.
Item 102: The oligosaccharide preparation for use in treatment, amelioration and/or prophylaxis of a bird suffering from necrotic enteritis and/or in risk thereof of any one of items 86-101, wherein the oligosaccharide preparation has a DP5 fraction content from 0.1 to 15% by relative abundance.
Item 103: The oligosaccharide preparation for use in treatment, amelioration and/or prophylaxis of a bird suffering from necrotic enteritis and/or in risk thereof of any one of items 86-102, wherein the ratio of DP2 fraction to DP1 fraction of the oligosaccharide preparation is 0.02-0.40 by relative abundance.
Item 104: The oligosaccharide preparation for use in treatment, amelioration and/or prophylaxis of a bird suffering from necrotic enteritis and/or in risk thereof of any one of items 86-103, wherein the ratio of DP3 fraction to DP2 fraction of the oligosaccharide preparation is 0.01-0.30 by relative abundance.
Item 105: The oligosaccharide preparation for use in treatment, amelioration and/or prophylaxis of a bird suffering from necrotic enteritis and/or in risk thereof of any one of items 86-104, wherein the aggregate content of DP1 and DP2 fractions in the oligosaccharide preparation is less than 50, 30, or 10% by relative abundance.
Item 106: The oligosaccharide preparation for use in treatment, amelioration and/or prophylaxis of a bird suffering from necrotic enteritis and/or in risk thereof of any one of items 86-105, wherein the oligosaccharide preparation comprises at least 103, 104, 105, 106 or 109 different oligosaccharide species.
Item 107: The oligosaccharide preparation for use in treatment, amelioration and/or prophylaxis of a bird suffering from necrotic enteritis and/or in risk thereof of any one of items 86-106, wherein two or more independent oligosaccharides of the oligosaccharide preparation comprise different anhydro-subunits.
Item 108: The oligosaccharide preparation for use in treatment, amelioration and/or prophylaxis of a bird suffering from necrotic enteritis and/or in risk thereof of any one of items 86-107, wherein the oligosaccharide preparation comprises one or more anhydro-subunits that are products of reversible thermal dehydration of monosaccharides.
Item 109: The oligosaccharide preparation for use in treatment, amelioration and/or prophylaxis of a bird suffering from necrotic enteritis and/or in risk thereof of any one of items 86-108, wherein the oligosaccharide preparation comprises one or more anhydro-glucose, anhydro-galactose, anhydro-mannose, anhydro-allose, anhydro-altrose, anhydro-gulose, anhydro-indose, anhydro-talose, anhydro-fructose, anhydro-ribose, anhydro-arabinose, anhydro-rhamnose, anhydro-lyxose, or anhydro-xylose subunits.
Item 110: The oligosaccharide preparation for use in treatment, amelioration and/or prophylaxis of a bird suffering from necrotic enteritis and/or in risk thereof of any one of items 86-109, wherein the oligosaccharide preparation comprises one or more anhydro-glucose, anhydro-galactose, anhydro-mannose, or anhydro-fructose subunits.
Item 111: The oligosaccharide preparation for use in treatment, amelioration and/or prophylaxis of a bird suffering from necrotic enteritis and/or in risk thereof of any one of items 86-110, wherein the oligosaccharide preparation comprises one or more 1,6-anhydro-β-D-glucofuranose or 1,6-anhydro-β-D-glucopyranose subunits. In some embodiments, the oligosaccharide preparation comprises both 1,6-anhydro-β-D-glucofuranose and 1,6-anhydro-β-D-glucopyranose anhydro-subunits.
Item 112: The oligosaccharide preparation for use in treatment, amelioration and/or prophylaxis of a bird suffering from necrotic enteritis and/or in risk thereof of any one of items 86-112, wherein a ratio of 1,6-anhydro-β-D-glucofuranose to 1,6-anhydro-β-D-glucopyranose is from about 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in the oligosaccharide preparation.
Item 113: The oligosaccharide preparation for use in treatment, amelioration and/or prophylaxis of a bird suffering from necrotic enteritis and/or in risk thereof of any one of items 86-112, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 within the oligosaccharide preparation.
Item 114: The oligosaccharide preparation for use in treatment, amelioration and/or prophylaxis of a bird suffering from necrotic enteritis and/or in risk thereof of any one of items 86-113, wherein the ratio of 1,6-anhydro-β-D-glucofuranose to 1,6-anhydro-β-D-glucopyranose is about 2:1 in the oligosaccharide preparation.
Item 115: The oligosaccharide preparation for use in treatment, amelioration and/or prophylaxis of a bird suffering from necrotic enteritis and/or in risk thereof of any one of items 86-114, wherein the ratio of 1,6-anhydro-β-D-glucofuranose to 1,6-anhydro-β-D-glucopyranose is about from 10:1 to 1:10, 9:1 to 1:10, 8:1 to 1:10, 7:1 to 1:10, 6:1 to 1:10, 5:1 to 1:10, 4:1 to 1:10, 3:1 to 1:10, 2:1 to 1:10, 10:1 to 1:9, 10:1 to 1:8, 10:1 to 1:7, 10:1 to 1:6, 10:1 to 1:5, 10:1 to 1:4, 10:1 to 1:3, 10:1 to 1:2, or 1:1 to 3:1 in each fraction of the oligosaccharide preparation.
Item 116: The oligosaccharide preparation for use in treatment, amelioration and/or prophylaxis of a bird suffering from necrotic enteritis and/or in risk thereof of any one of items 86-115, wherein the ratio of 1,6-anhydro-β-D-glucofuranose to 1,6-anhydro-β-D-glucopyranose is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:8, 1:9, or 1:10 in each fraction of the oligosaccharide preparation.
Item 117: The oligosaccharide preparation for use in treatment, amelioration and/or prophylaxis of a bird suffering from necrotic enteritis and/or in risk thereof of any one of items 86-116, wherein the ratio of 1,6-anhydro-β-D-glucofuranose to 1,6-anhydro-β-D-glucopyranose is about 2:1 in each fraction of the oligosaccharide preparation.
Item 118: The oligosaccharide preparation for use in treatment, amelioration and/or prophylaxis of a bird suffering from necrotic enteritis and/or in risk thereof of any one of items 86-117, wherein at least 1%, 5%, 10%, 20%, 30%, 40%, 50%, 60%, 70%, 80%, or 90% of anhydro-subunits in the oligosaccharide preparation are selected from a group consisting of 1,6-anhydro-β-D-glucofuranose and 1,6-anhydro-β-D-glucopyranose.
Item 119: The oligosaccharide preparation for use in treatment, amelioration and/or prophylaxis of a bird suffering from necrotic enteritis and/or in risk thereof of any one of items 86-118, wherein the weight average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol.
Item 120: The oligosaccharide preparation for use in treatment, amelioration and/or prophylaxis of a bird suffering from necrotic enteritis and/or in risk thereof of any one of items 86-119, wherein the number average molecular weight of the oligosaccharide preparation is about from 300 to 5000 g/mol, 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1000 g/mol, 400 to 900 g/mol, 400 to 800 g/mol, 500 to 900 g/mol, or 500 to 800 g/mol.
Item 121: The oligosaccharide preparation for use in treatment, amelioration and/or prophylaxis of a bird suffering from necrotic enteritis and/or in risk thereof of any one of items 86-120, wherein the distribution of the degree of polymerization is determined and/or detected by MALDI-MS, GC-MS, LC-MS, SEC, HPLC and/or combination(s) thereof (e.g. MALDI-MS and SEC).
Item 122: The oligosaccharide preparation for use in treatment, amelioration and/or prophylaxis of a bird suffering from necrotic enteritis and/or in risk thereof of any one of items 86-121, wherein the degree of polymerization of the oligosaccharide preparation may be determined based on its molecular weight and molecular weight distribution.
Item 123: The oligosaccharide preparation for use in treatment, amelioration and/or prophylaxis of a bird suffering from necrotic enteritis and/or in risk thereof of any one of items 86-122, wherein the oligosaccharide preparation is comprised in the nutritional composition at a concentration of at least 50 g per ton of feed (e.g. at least 70 g, 100 g, 200 g, 300 g, 400 g, 500 g, 600 g, 700 g, 800 g, 900 g per ton of feed); and/or wherein the oligosaccharide preparation comprised in the nutritional composition at an inclusion rate of at least 50 ppm (e.g. at least 50, 70, 100, 150, 200, 300, 400, 500 ppm); and/or wherein the oligosaccharide preparation comprised in the nutritional composition at a concentration of at least 50 ppm (e.g. at least 50, 70, 100, 150, 200, 300, 400, 500 ppm).
Item 124: The oligosaccharide preparation for use in treatment, amelioration and/or prophylaxis of a bird suffering from necrotic enteritis and/or in risk thereof of any one of items 86-123, wherein the oligosaccharide preparation is administered for at least one day, preferably for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, or 245 days, most preferably, the nutritional composition is administered continuously, i.e. uninterruptedly.

The oligosaccharide preparation referred to herein may be characterized by any one, two or more or even all of the individual oligosaccharide preparation features as described in the items above. In other words, the oligosaccharide preparation may be characterized by any combination of the individual features of the oligosaccharide preparation described in the items above. For instance, in a particular embodiment of a method or use according to the invention, the oligosaccharide preparation may be characterized by a combination of the combined oligosaccharide preparation features as described in items 9, 11, 20 and 39.

In the following, the present invention is further described by non-limiting examples. The present invention as disclosed herein is not limited to specific embodiments, methodology, examples, protocols etc. described herein but solely defined by the claims.

### EXAMPLES

### Example 1—Effect of Oligosaccharide Preparation on Birds Subjected to Necrotic Enteritis Challenge

A feeding trial was performed to study the effects of oligosaccharide preparations on zootechnical and biological parameters of broiler chickens undergoing a necrotic enteritis challenge model created by Eimeria maxima and Clostridium perfringens inoculation.

Oligosaccharide preparations were prepared as disclosed in WO 2020/097458 and WO 2016/007778, in particular in any one of Examples 1-7, 16-18 of WO 2020/097458 A1, in the methods described in paragraph [317], and/or in any one of Examples 73-77, 80-89, 97-99, 101-110 of WO 2016/007778 A1. The oligosaccharide preparations comprise at least n fractions of oligosaccharides each having a distinct degree of polymerization selected from 1 to n (DP1 to DPn fractions), wherein n is an integer greater than 3; wherein each of a DP1 and DP2 fraction independently comprises from about 0.5% to about 15% of anhydro-subunit containing oligosaccharides by relative abundance as determined by mass spectrometry. In addition, the prepared oligosaccharide preparations can be characterized by the features described herein.

For the feeding trial, 1,600 birds were allotted to floor pens with 25 birds per pen. Treatments were replicated in eight blocks, randomized within blocks of 8 pens each. Birds were allotted to pens to achieve equal start pen weights across treatments. One pen was considered the experimental unit.

Trial duration was 42 days. All birds received vaccination against coccidiosis (COCCIVAC®-B52 by Merck Animal Health USA, a live oocysts vaccine isolated from chickens, prepared from anticoccidial-sensitive strains of E. acervulina, E. maxima, E. maxima MFP, E. mivati, and E. tenella according to the product bulletin).

A negative control group (NC) was not subjected to necrotic enteritis challenge, and was not administered the oligosaccharide preparation. A challenge control group (CC) was subjected to necrotic enteritis challenge, but not administered the oligosaccharide preparation. A first test group (OP500) was subjected to necrotic enteritis challenge, and was administered feed comprising 500 ppm of the oligosaccharide preparation. A second test group (OP700) was subjected to necrotic enteritis challenge, and was administered feed comprising 700 ppm of the oligosaccharide preparation. A third test group (OP900) was subjected to necrotic enteritis challenge, and was administered feed comprising 900 ppm of the oligosaccharide preparation.

Animal management: Birds were of the following demographics: Genetics: Cobb 500; Sex: Male; Starting age: day of hatch; Only birds that appear healthy were used in this study.

The temperature of the building was monitored. Environmental conditions during the trial (temperature) were appropriate (optimum) to the age of the animals. Illumination was provided by fluorescent bulbs placed above the pens. The lighting scheme was 22 light and 2 dark hours per day. Standard floor pen management practices were used throughout the experiment. Animals and housing facilities were inspected twice daily, observing, and recording the general health status, constant feed and water supply as well as temperature, removing all dead birds, and recognizing unexpected events.

Pens were checked daily for mortality. A bird was culled only to relieve suffering. When a bird was culled or found dead, the pen, date and removal weight (kg) were recorded. A gross necropsy was performed on all dead or culled birds to determine the probable cause of death.

Feed and water: Quantities of test article/s were documented for each batch manufactured. Feed was mixed to assure a uniform distribution of test article/s. Experimental feeds were prepared from uniform basal diets. All treatment feeds were mixed with a Davis S-20 mixer and pelleted using a California Pellet Mill. Broiler diets were fed as crumbles (starter feed) and as pellets (grower and finisher). Diet formulations were included in the source data. Feed allotment=5 kg/bird×200 birds/trt=1,000 kg. The diets were provided ad libitum in one tube-type feeder per pen. From day 1 until day 7, feed was also supplied on a feeder tray, placed on the litter of each pen. Water was provided ad libitum. All feed was weighed by pen. Starter feed was fed from Day 0 to 21. On Day 14, non-consumed starter was weighed back and re-issued. On Day 21, non-consumed starter was weighed and discarded. Grower feed was issued and fed until Day 35. On Day 35, non-consumed Grower was weighed and discarded. Finisher feed was issued and fed until Day 42. Diet formulation is shown in Table 1 below.

TABLE 1
Diet formulation.
Diet formulation, % as-fed

| Ingredient | Starter % | Grower % | Finisher % |
|---|---|---|---|
| CORN, YELLOW, GRAIN | 56.36 | 60.76 | 68.92 |
| SOYBEAN MEAL | 39.31 | 34.37 | 26.45 |
| FAT, VEGETABLE | 1.33 | 2.14 | 2.15 |
| CALCIUM CARBONATE | 1.05 | 1.05 | 0.95 |
| DICALCIUM PHOSPHATE. | 0.97 | 0.82 | 0.63 |
| Methionine MHA | 0.32 | 0.22 | 0.24 |
| SALT, PLAIN (NaCl) | 0.29 | 0.29 | 0.3 |
| L YSINE | 0.18 | 0.19 | 0.21 |
| Trace Mineral | 0.08 | 0.08 | 0.08 |
| Vitamin premix | 0.07 | 0.05 | 0.05 |
| L-Threonine 98.5 | 0.03 | 0.01 | 0.01 |
| quantum blu | 0.01 | 0.01 | 0.01 |

Calculated analysis

| Calculate Analysis | Starter | Grower | Finisher |
|---|---|---|---|
| CP, % | 22.00 | 20.00 | 17.00 |
| Fat, % | 3.61 | 4.50 | 4.68 |
| Fiber, % | 2.23 | 2.16 | 2.07 |
| Calcium, % | 0.90 | 0.85 | 0.76 |
| Total P, % | 0.57 | 0.52 | 0.46 |
| aP, % | 0.45 | 0.42 | 0.38 |
| ME Poultry | 2,992 | 3,067 | 3,116 |
| Na, % | 0.18 | 0.18 | 0.18 |
| dig Met, % | 0.56 | 0.46 | 0.45 |
| dig Cys, % | 0.29 | 0.28 | 0.26 |
| dig Lys, % | 1.18 | 1.08 | 0.93 |
| dig Thr, % | 0.76 | 0.68 | 0.59 |
| dig Iso, % | 0.87 | 0.79 | 0.66 |
| dig Val, % | 0.9 | 0.83 | 0.72 |
| dig TSAA, % | 0.88 | 0.76 | 0.72 |

Mineral Premix - added at a rate of 1.5 lb per ton of feed.

| | |
|---|---|
| Calcium (Ca) Min. | 3.20% |
| Calcium (Ca) Max. | 4.20% |
| Iron (Fe) Min. | 2.63% |
| Magnesium (Mg) Min. | 2.68% |
| Manganese (Mn) Min. | 13.40% |
| Zinc (Zn) Min | 10.70% |
| Copper (Cu) Min | 4000 ppm |
| lodine (I) Min. | 1000 ppm |
| Selenium (Se) Min. | 400 ppm |

Vitamin Premix - added at a rate of 1.0 lb per ton of feed.

| | |
|---|---|
| Vitamin A, I..U./LB | 1,000,000 |
| Vitamin D3, I..U./LB | 200,000 |
| Vitamin E, I..U./LB | 2,000 |
| Vitamin B-12, MG./LB | 2.20 |
| Riboflavin, MG./LB 800 | 800 |
| Niacin, MG./LB 8 | 8,000 |
| d-Pantothenic Acid, MG./LB | 2,000 |
| Choline, MG./LB 34, 72 | 34,720 |
| Menadione, MG./LB 132 | 132 |
| Folic Acid, MG./LB | 100 |
| Thiamine, MG./LB | 400 |
| Pyridoxine, MG./LB | 400 |
| Biotin, MG./LB | 20 |
| Ethoxyquin, MG./LB | 23,000 |

Trial procedures: Three feed samples (~2 lb each) were collected: one each from the beginning, middle, and end of the batch of treatment diet and mixed to form a composite sample (2 lb). The composite sample was split into 3 aliquots. One composite sample of each trt/diet was held for 30 days after the end of the study and the other two were used for analysis of dietary composition.

Necrotic enteritis challenge model (expected NE mortality=5-10%): All pens except NC were subjected to necrotic enteritis challenge by the example of confrontation with Eimera and C. perfringens. On day 14, 5,000 Eimeria maxima oocysts per bird were administered. This isolate has come from the field and has been identified as providing a weak challenge. Oocyst inoculum was mixed into the feed at the base of the tube feeders. On Day 19, 20, and 21, all were given a broth culture of Clostridium perfringens (CP6). This field isolate originated from a commercial broiler operation. Fresh inoculum was used each day with a titration level of approximately 1.0×10⁸⁻⁹/bird. Inoculum was administered by mixing into the feed at the base of the tube feeders. Number of days that the CP is given was depended on how much mortality is observed daily. Three days of dosing was used.

Coccidia oocysts per gram litter (OPG): On Day 0 a baseline litter sample was collected for baseline OPG. On day 14, 21, 28, and 35 fresh fecal samples were collected from each pen. These representative samples were tested to determine the degree of oocysts shedding/cycling. Oocysts per gram was determined for each sample.

Necropsy sample collection: On Day 21, 2 birds/pen were randomly selected, sacrificed, individually weighted, and exampled for presence of necrotic enteritis (NE) lesions. From each bird, a section of the ileum was collected into formalin. Formalin was replaced with ethanol 24 hours later and these fixed samples were analyzed for histology based on need determined after analysis of performance data. Similarly, ceca samples were collected and stored in −80° C. and analysis determined after reviewing performance data.

Pen weights and feed disappearance: Pen weights and feed disappearance were recorded on day 14, 21, 35, and 42. Feed conversion ratio was calculated.

Mortalities: Birds found dead during the study were noted and were not replaced. Pen number, the date of mortality, sex, weight, and diagnosis were recorded. No birds were replaced during the course of the study.

Data analysis: Outcome variables were i) Performance (pen and feeder weights on day 14, 21, 35, and 42. Cumulative BWG and FCR were calculated); ii) OPGs (oocysts per gram feces); iii) Mortality (number of birds confirmed with NE-related and total mortality); iv) Lesion Scores.

Data and analysis: Means for live weight, weight gain, feed consumption, feed conversion ratio (FCR), OPGs, and mortality (total and NE) were calculated. Dara were analyzed by ANOVA and then means separated by Tukey's test (P<0.05) by using JMP data analysis software.

Results: Considering the overall experimental period, 0 to 42 days of age, supplementation with the oligosaccharide preparation improved the body weight gain (BWG) and feed conversion ratio (FCR) of the birds when compared to the challenged control group (CC). Necrotic enteritis-challenged groups that were administered the oligosaccharide preparation (OP500, OP, 700, OP900) were statistically similar to the non-challenged control group NC. Necrotic enteritis (NE)-related mortality was higher in the CC group compared to the NC group. Administration of the oligosaccharide preparation was found to reduce NE-related mortality compared to the CC group. The lesion score characteristic of NE was higher in the CC group than in any of the other groups. Notably, groups to which the oligosaccharide preparation was administered had a reduced lesion score compared to the CC group. In particular, the necrotic enteritis-challenge promoted a higher frequency of birds with lesion scores 1 and 2 and a drastic reduction in lesion scores 0 in the CC group compared to the NC group. However, groups receiving the oligosaccharide preparation (OP500, OP700, OP900) reduced the number of birds with lesions 1 and 2 and increased the frequency of birds with lesions 0. Thus, administration of the oligosaccharide preparation was found to lead to a reduction in severe lesions despite NE-challenge. Results are summarized in Table 2 below.

In conclusion, administration of the oligosaccharide preparation in all groups was found beneficial for reducing the negative effects of necrotic enteritis in birds. Administration of the oligosaccharide preparation at 900 ppm was even found to completely restore the growth performance of challenged birds and to reduce NE-related mortality similar to non-challenged birds.

TABLE 2
Body weight gain (BWG), feed intake (FI), feed conversion ratio (FCR), NE-related mortality
(NE-mortality), foot pad lesions (FPD), and lesion score of broiler chickens.

| | Treatment | | | | | | | | | | | | P value |
|---|---|---|---|---|---|---|---|---|---|---|---|---|---|
| | group: | NC | | CC | | OP500 | | OP700 | | OP900 | | SEM | treat |
| 1-14 | BWG, g | 320 | | 312 | | 311 | | 307 | | 324 | | 7.7 | 0.78 |
| days | FI, g | 395 | | 382 | | 373 | | 378 | | 391 | | 10.2 | 0.73 |
| | FCR | 1.238 | | 1.223 | | 1.199 | | 1.252 | | 1.242 | | 0.020 | 0.17 |
| 1-21 | BWG, g | 647 | a | 562 | b | 637 | ab | 631 | ab | 692 | a | 21.5 | 0.001 |
| days | FI, g | 2,850 | ab | 2,797 | ab | 2,774 | b | 2,826 | ab | 2,909 | ab | 30.4 | 0.006 |
| | FCR | 1.470 | b | 1.638 | a | 1.453 | b | 1.464 | b | 1.406 | b | 0.030 | <0.0001 |
| 1-35 | BWG, g | 1,768 | a | 1,635 | b | 1,718 | ab | 1,747 | ab | 1,798 | a | 30.2 | 0.0003 |
| days | FI, g | 2,850 | | 2,797 | | 2,774 | | 2,826 | | 2,909 | | 30.4 | 0.006 |
| | FCR | 1.613 | ab | 1.710 | a | 1.618 | ab | 1.628 | b | 1.617 | b | 0.020 | <0.0001 |
| 1-42 | BWG, g | 2,648 | a | 2,410 | b | 2,599 | a | 2,605 | a | 2,677 | a | 44.8 | <0.0001 |
| days | FI, g | 4,589 | | 2,506 | | 4,456 | | 4,520 | | 4,612 | | 47.5 | 0.2 |
| | FCR | 1.735 | b | 1.884 | a | 1.716 | b | 1.727 | b | 1.725 | b | 0.024 | <0.0001 |
| | NE-mortality, % | 0.0 | b | 5.5 | a | 0.5 | ab | 1.5 | ab | 0.5 | ab | 1.10 | 0.001 |
| | FPD, % | 1.25 | ab | 0.89 | b | 1.25 | ab | 1.98 | a | 1.30 | ab | 0.20 | 0.02 |
| | Lesion score | 0.0 | b | 1.2 | a | 0.58 | a | 0.50 | ab | 0.38 | ab | 0.15 | <0.0001 |

1. Method for reducing necrotic enteritis effect in a bird, comprising the step of administering an oligosaccharide preparation to a bird in need of reducing necrotic enteritis effect,
wherein the oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90%, e.g. from 1% to 90%, or e.g. from about 0.5% to about 15%, of anhydro-subunit containing oligosaccharides by relative abundance as determined by mass spectrometry.

2. The method of claim 1, wherein the necrotic enteritis effect is at least one selected from the group of reduced body weight gain of the bird in need of reducing necrotic enteritis effect compared to a control bird not in need of reducing necrotic enteritis effect; increased feed conversion ratio of the bird in need of reducing necrotic enteritis effect compared to the control bird; increased mortality of the bird in need of reducing necrotic enteritis effect compared to the control bird; and increased lesion score of the bird in need of reducing necrotic enteritis effect compared to the control bird.

3. The method of claim 1, wherein the bird in need of reducing necrotic enteritis effect is challenged by Clostridium perfringens.

4. The method of claim 1, wherein the oligosaccharide preparation is comprised in a nutritional composition administered to the bird.

5. The method of claim 1, wherein the oligosaccharide preparation is comprised in the nutritional composition at an inclusion rate of at least 50 ppm, e.g. at least 50, 70, 100, 150, 200, 300, 400, 500, 600, 700, 800, 900 ppm.

6. The method of claim 1, wherein the oligosaccharide preparation is administered to the bird before, after, and/or simultaneously with the diet of the bird in need of reducing necrotic enteritis effect, preferably simultaneously with the diet.

7. The method of claim 1, wherein the bird is poultry, e.g. chicken, duck, goose, turkey, guinea fowl, pigeon, quail; preferably chicken.

8. The method of claim 1, wherein the necrotic enteritis effect is reduced on at least one day within 42 days after a first administration of the oligosaccharide preparation, selected from the group of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 day(s).

9. The method of claim 1, wherein n of the oligosaccharide preparation is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

10. The method of claim 1, wherein at least one fraction of the oligosaccharide preparation comprises less than 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, or 2% anhydro-subunit containing oligosaccharides by relative abundance; and/or wherein each fraction of the oligosaccharide preparation comprises greater than 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, or 80% anhydro-subunit containing oligosaccharides by relative abundance.

11. The method of claim 1, wherein the oligosaccharide preparation has a weight average molecular weight from about 300 to 5000 g/mol, e.g. from about 2000 to 2800 g/mol, 2100 to 2700 g/mol, 2200 to 2600 g/mol, 2300 to 2500 g/mol, or 2320 to 2420 g/mol; 500 to 5000 g/mol, 700 to 5000 g/mol, 500 to 2000 g/mol, 700 to 2000 g/mol, 700 to 1500 g/mol, 300 to 1500 g/mol, 300 to 2000 g/mol, 400 to 1300 g/mol, 400 to 1200 g/mol, 400 to 1100 g/mol, 500 to 1300 g/mol, 500 to 1200 g/mol, 500 to 1100 g/mol, 600 to 1300 g/mol, 600 to 1200 g/mol, or 600 to 1100 g/mol; and/or wherein the oligosaccharide preparation has a number average molecular weight from about 1000 to 2000 g/mol, 1100 to 1900 g/mol, 1200 to 1800 g/mol, 1300 to 1700 g/mol, 1400 to 1600 g/mol, or 1450 to 1550 g/mol.

12. The method of claim 1, wherein the relative abundance of oligosaccharides in each of the n fractions of the oligosaccharide preparation decreases monotonically with its degree of polymerization.

13. Use of an oligosaccharide preparation for reducing necrotic enteritis effect in a bird in need thereof,
wherein the oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90%, e.g. from 1% to 90%, or e.g. from about 0.5% to about 15%, of anhydro-subunit containing oligosaccharides by relative abundance as determined by mass spectrometry.

14. Oligosaccharide preparation for use in treatment, amelioration and/or prophylaxis of a bird suffering from necrotic enteritis or in risk thereof,
wherein the oligosaccharide preparation comprises at least n fractions of oligosaccharides each having a distinct degree of polymerization selected from 1 to n (DP1 to DPn fractions), wherein n is an integer greater than or equal to 2; and
wherein each fraction comprises from at least about 0.5% to about 90%, e.g. from 1% to 90%, or e.g. from about 0.5% to about 15%, of anhydro-subunit containing oligosaccharides by relative abundance as determined by mass spectrometry.
